(19) United States
(12) Patent Application Publication
(10) Pub. No.: US 2015/0096475 A1
(43) Pub. Date: Apr. 9, 2015

(54) SYSTEM AND METHODS FOR PROVIDING PROTECTIVE COVERAGE OF AN OPERATIONAL SURFACE

(71) Applicants: Seon-Kyu Lee, Chicago, IL (US); Stewart Thomas, Chicago, IL (US)

(72) Inventors: Seon-Kyu Lee, Chicago, IL (US); Stewart Thomas, Chicago, IL (US)

(21) Appl. No.: 14/508,241

(22) Filed: Oct. 7, 2014

Related U.S. Application Data
(60) Provisional application No. 61/887,680, filed on Oct. 7, 2013.

(51) Int. Cl. A61B 19/02; A47B 97/00; A61B 19/00

(52) U.S. Cl. CPC A61B 19/0248; A61B 19/38; A47B 97/00; A61B 2019/0255; A61B 2017/00477
USPC 108/50.11

(57) ABSTRACT

The present invention relates generally to a system and methods for providing protective coverage of an operational surface and any items that may be on the surface. Certain embodiments of the system may be used to help to maintain the sterility of a surface and the items on the surface. Advantageously, such embodiments of the system may be used to maintain the sterility of surgical instruments and materials, and the table surface on which they are placed prior to the start of the surgical procedure. Other embodiments of the system may be used to at least help to prevent the contamination of or maintain the freshness of food and the containers and utensils used to hold, prepare, and serve the food.

This application claims the benefit of U.S. Provisional Application No. 61/887,680 filed on Oct. 7, 2013, which is incorporated by reference in its entirety.

### FIELD OF THE INVENTION

The present invention relates generally to a system and methods for providing protective coverage of an operational surface. More specifically, the invention is directed to a system that includes a protective cover and an attachment component through the use of which the cover may be secured relative to the operational surface to provide coverage of the surface and any items that may be on the surface. Certain embodiments of the system may be used to help to maintain the sterility of a surface and the items on the surface. Advantageously, such embodiments of the system may be used to maintain the sterility of surgical instruments and materials, and the table surface on which they are placed prior to the start of the surgical procedure. Other embodiments of the system may be used to at least help to prevent the contamination of or maintain the freshness of food and the containers and utensils used to hold, prepare, and serve the food.

### BACKGROUND OF THE INVENTION

In a variety of contexts, items are placed on table tops as a step in or in preparation for and in order to facilitate some other procedure.

One such context is a medical operating room and the surgical procedures conducted there. In order to conduct a surgical procedure, a surgical team typically needs ready access to a variety of surgical instruments, such as scalpels, forceps, scissors, claims, retractors, and the like. In order that the surgical team may have such ready access, the surgical instruments and possibly other materials are placed on a surface that is positioned within easy reach of the team members. Often this surface is the top of a table. The table is often movable so that it can be repositioned according to the needs of the team during the course of surgery.

The surgical instruments are often not placed directly onto the surface of the table. Instead, one or more sterilized covers may be placed onto the surface of the table and the instruments and materials needed for the surgery positioned on the upper surface of the covers. In such cases, the one or more sterilized covers act as under covers.

The above described steps define a certain portion of the routine work-flow that takes place before many surgical procedures. However, this procedure of preparing the one or more tables that are used to hold the instruments and materials that are needed during surgery takes time. The additional time required to prepare the surgical instrument table with instruments is commonly referred to as “mandatory” preparation time within the profession. “Pre-preparing” the surgical instrument table before surgery is currently not recommended when the patient on which the surgery is to be conducted is human largely because there is no way for the surgical team to be confident that the surgical instruments are still sterile once they are placed on the surgical instrument table and remain there for a length of time. In other words, if the surgical instrument table was “pre-prepared” prior to a scheduled surgery, it is possible that the sterility of the instruments placed on the table may be compromised during the period of time between when the surgical instrument table was prepared and when the surgical instruments are actually used to perform a surgical procedure. The use of non-sterile surgical instruments could lead to undesirable surgical complications such as infections. Simply placing a sterilized cover of the same type that may be used as the table under cover over the “pre-prepared” instrument table would not solve the problem. Such sterilized covers are often made from materials that are opaque. As a result, a person could not confirm without lifting the opaque cover whether the proper instruments had been laid out on the table in the position and order required by the surgical team for the given procedure.

Another context is the food industry. Often times, a variety of items including vegetables, meats, sauces, and spices are needed to prepare dishes. If such items are prepared in advance, they are often times individually covered. With the initiation of and during the preparation of the dishes, these covers must be individually removed from each of the containers. The covers are placed back onto the containers as the use of each of the items is completed to maintain the freshness and prevent the contamination of the contained items. Such contamination may come not only from unintended materials from falling into the open containers but also the odors and smells produced by the cooking or processing of other food items. This process of removing the covers and returning the covers to the containers takes time which slows down the process of making the dishes. Such time is often in short supply especially in busy restaurant kitchens. Also, space must be provided for the covers as they are removed so that they can be easily reached by the chef and placed back onto the proper container. Such space—within arms' reach of the chef preparing a dish—is often also in short supply in commercial kitchens. Also, the covers removed from the containers must be placed in some order so that a busy chef can easily find the correct cover for a container.

In certain contexts, dishes are prepared in advance and placed on one or more tables before those for whom the dishes are prepared actually are given access to the dishes. These dishes are often individually covered. A buffet table is one example of such an arrangement. The covers are removed just prior to the time the diners are given access to the dishes. This process of removing the covers of all the dish containers prior to allowing diners to access the dishes is a time consuming process. Also, space must be provided on which the removed covers can be positioned as they are removed. This space must be separate and apart from the immediate area in which the dishes are positioned in order to maintain the aesthetics of the area and prevent a diner from coming in unintended contact with the covers.

When food is to be offered and consumed outside, such as at a barbeque or picnic, components for meals and individual dishes are often placed on a variety of support surfaces—or example, wooden picnic tables, folding tables, and the like—and individually covered to prevent insects from gaining access to the dishes, or the dishes becoming contaminated, and otherwise to maintain the integrity and freshness of the dishes. As above, in order that the food can be used to prepare other dishes or meals or served, the covers must be removed and placed in some order and location so that the covers do not become contaminated and, when needed, can be easily located and attached to the containers. It is not unusual in an outdoor setting to have no such readily available place to store such covers during the course of a barbecue or a picnic.

From the above, it is clear that there is a demand for a system and methods through the use of which a surface, on which one or more items are positioned, may be covered to preserve the sterility or freshness, or prevent the contamination of the surface and the items. The present invention satisfies the demand.

### SUMMARY OF THE INVENTION

The present invention is directed to a system and methods for providing protective coverage of an operational surface and the items placed on the surface. For purposes of this application, an operational surface is any apparatus or device that may include some component or have one or more items placed on the surface that may be useful for some procedure. Certain embodiments of the present invention is directed to a system that includes a flexible cover and an attachment component through the use of which the cover may be secured relative to a surface to provide protective coverage of the surface and that which is positioned on or through the surface.

Certain embodiments of the system may be used to help to maintain the sterility of an operational surface and the items on the surface. Such embodiments of the system may be used to maintain the sterility of the surface of a surgical instrument table on which the instruments, items, and materials that may be used during the surgery may be placed. Certain specific embodiments of the present invention that may be used for such a surgical application include a protective cover that may be secured to the surface of, for example, a surgical table through the use of an attachment component in order to maintain the sterility of the surface and that which is placed on it. Another embodiment of the present invention may include a protective cover having an attachment component that may be securable to the upper surface of the one or more separate sterile covers positioned on the surface of the surgical table on which the surgical instruments and materials are placed. An example of such a separate cover to which the protective cover of the present invention may be secured is a conventional sterile cover used as an under cover for the surgical instruments and materials.

The protective cover may include one or more forms of attachment components. One embodiment of an attachment component includes an adhesive surface positioned on one side of the protective cover. The adhesive surface may be maintained by a peel-off layer that is removable in order to expose the adhesive as needed to secure the cover to the operational surface. Certain embodiments of the adhesive surface may include an adhesive or other material and/or may be configured so that it is evident that at least the adhesive component has been disturbed and/or the protective cover has been at least partially opened. Such embodiments are advantageous when it is critical to know whether the operational surface has been exposed prior to a procedure and the sterility or freshness possibly compromised.

Another embodiment of the present invention includes an attachment component that facilitates a mechanical connection of the protective cover to or adjacent to the operational surface. One example of such an attachment component that facilitates such mechanical connection includes a closure mechanism that includes one or more male members that may be received in female members. In certain embodiments, the one or more male members may be positioned on a surface of the protective cover and received in an appropriately aligned and number of female members positioned on or adjacent to the surface of the operational surface. One such embodiment includes the one or more female members positioned on the surface of a flexible tape, the reverse side of which includes a covered adhesive component. By exposing the adhesive, the tape may be secured to the operational surface or the sterile under cover in a position to facilitate the easy insertion of the male members aligned on a surface of the protective cover. An additional embodiment includes a table surface which includes one portion of the closure mechanism—for example, the female shaped component—formed in the table surface. Such an embodiment is advantageous in that the additional time to add the female component to the surface of the table or the sterile under cover is rendered unnecessary. Such a component may be configured and formed of a material such that it may be easily sterilized when the entire operational surface is sterilized. Other embodiments include a reversed arrangement in which the one or more female members are positioned on a surface of the protective cover and the one or more male members are positioned on the surface or adjacent to the surface or formed in the operational surface. By pressing the male members into the female members the protective cover may secured to and protectively cover the operational surface. Embodiments of the present invention that include a mechanical connection attachment component are advantageous in applications in which the protective cover may need to opened and closed more than once. Applications involving food preparation or food service are such applications. A chef may need to open the protective cover to gain access to one or more items needed to prepare a dish, then close the protective cover to preserve the freshness or prevent the contamination of the items collected on the operational surface.

Other embodiments of the present invention that permit the protective cover to be opened and closed more than once include an adhesive component in which the adhesive portion is a “low-tack”, reusable, pressure-sensitive adhesive. Such an adhesive allows the protective cover to be stuck to an object, easily lifted off the object, and re-stuck as needed.

Additional embodiments of the attachment component can include magnetic elements in which a magnet on the surface of the protective cover may be attached to the operational surface when the operational surface is of a composition to which the magnet may attach.

Such embodiments of the system that allow the protective cover to be opened and closed as needed may be used to at least help to prevent the contamination of or maintain the freshness of protect food and the containers and utensils used to hold, prepare, and serve the food. The need for multiple covered containers in which the multiple covers must be removed, stored, then replaced is thereby obviated.

Other embodiments of the present invention include a protective cover including an attachment component that is configured such that the cover may form a pouch in which the items or materials to be protected are enclosed. Additional embodiments of the present invention may be used in conjunction with each other such that a protective pouch is formable by joining two or more protective covers with each other through the attachment component on each. It is appreciated that two or more protective covers can be attached together so as to form a pouch that may contain one or more instruments or other equipment, such as a surgical microscope, that may not be necessarily on a table.

An advantage of certain embodiments of the present invention that allow sterile conditions to be maintained within the interior defined by the protective cover is that the “mandatory” preparation time for a surgical procedure may be eliminated altogether. The surgical instruments and materials needed by the surgical team can be laid out in advance and, through the use of certain embodiments of the present invention, the sterility of these items maintained until needed. Surgical teams can initiate emergency surgical procedures—such as intracranial arterial thrombectomy for acute stroke patients or emergency surgical interventions for life threatening bleeding—more quickly. Success rates may improve. Overall, surgical instrument tables that are “pre-prepared” through the use of the present invention can improve operating room efficiency through work-flow improvement and possibly decrease the cost of surgical procedures.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed for carrying out this invention, but that the invention will include all embodiments falling within the scope of the present application. Moreover, the advantages described herein are not necessarily the only advantages of the invention and it is not necessarily expected that every embodiment of the invention will achieve all of the advantages described.

### BRIEF DESCRIPTION OF THE DRAWINGS

FIG. 1A shows a perspective view of an embodiment of the present invention ready for deployment relative to an operational surface;

FIG. 1B shows a perspective view of another embodiment of the present invention deployed relative to an operational surface and including an under cover;

FIG. 1C shows a perspective view of a portion of the embodiment of the present invention shown in FIG. 1B;

FIG. 1D shows an overhead view of the embodiment shown in FIG. 1A;

FIG. 1E shows an overhead view of a portion of the embodiment shown in FIG. 1D;

FIG. 2A shows a perspective view of a portion of an attachment component;

FIG. 2B shows a perspective view of a portion of the embodiment illustrated in FIG. 1C deployed relative to an operational surface;

FIG. 3A shows a perspective view of a portion of an embodiment of the present invention that includes a tab to facilitate the opening of the protective cover;

FIG. 3B shows a side view of a portion of the embodiment of the present invention shown in FIG. 3A in which the tab is being used to open the protective cover;

FIG. 4A shows a perspective view of an embodiment of a restraint that may be used in certain embodiments of the present invention to secure the protective cover relative to an operational surface;

FIG. 4B shows a perspective view of the embodiment of the restraint shown in FIG. 4A positioned to secure the protective cover relative to an operational surface;

FIG. 4C shows a perspective view of the embodiment of the restraint shown in FIG. 4A and FIG. 4B in a securing position;

FIG. 5A shows a side view of an embodiment of the present invention in which a tab is being used to open the protective cover;

FIG. 5B shows a perspective view of an embodiment of the present invention in which the protective cover has been opened and showing the possible downward movement of the lower portion of one embodiment of the protective cover to thereby drape a portion of the table;

FIG. 5C shows a perspective view of the embodiment of the present invention shown in FIG. 5B in which the draping action of the lower portion of the protective cover has been largely completed;

FIG. 6 shows side views of various embodiments of the present invention and possible sizes;

FIG. 7A shows a cross section of an embodiment of the present invention in which a portion of an operational surface is configured to facilitate the mechanical attachment of a protective cover; and

FIG. 7B shows a cross section of another embodiment of the present invention in which the attachment component facilitates the mechanical connection of the protective cover relative to the operational surface.

### DETAILED DESCRIPTION

The present invention is directed to a system 31 and methods for providing protective coverage of an operational surface 21. Certain embodiments of the system 31 include a protective cover 51 and an attachment component 71 through the use of which the protective cover 51 may be secured relative to a surface to provide protective coverage of that which is positioned on or through the operational surface 21.

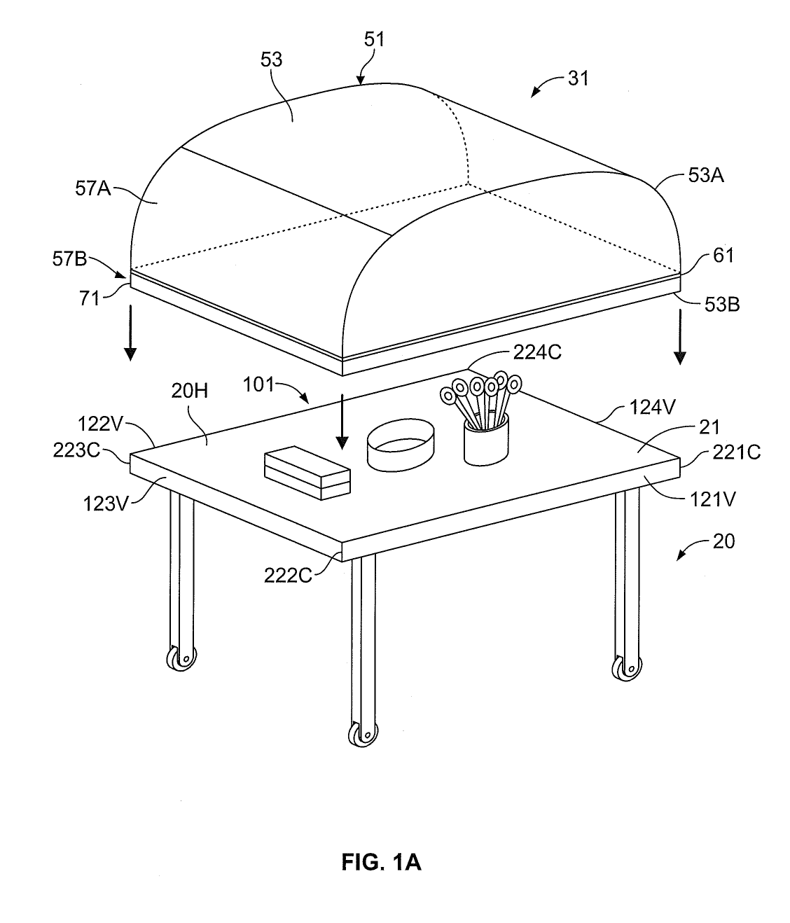

FIG. 1A provides a perspective view of one embodiment of the present invention in place for deployment relative to an operational surface 21. The operational surface 21 shown in FIG. 1A is the top horizontal surface 20H of a table 20. A table 20 with which the present invention may be used may be more made mobile with the use of leg casters such as the table 20 shown in FIG. 1A. The illustrated table 20 includes sets of opposing vertical surfaces 121V, 122V, 123V, 124V that meet at corners 221C, 222C, 223C, 224C. Certain items for which protection is sought by a user—collectively identified by the number 101—are positioned, for example, on the horizontal surface 20H of the table 20 in preparation for some additional procedure to be conducted.

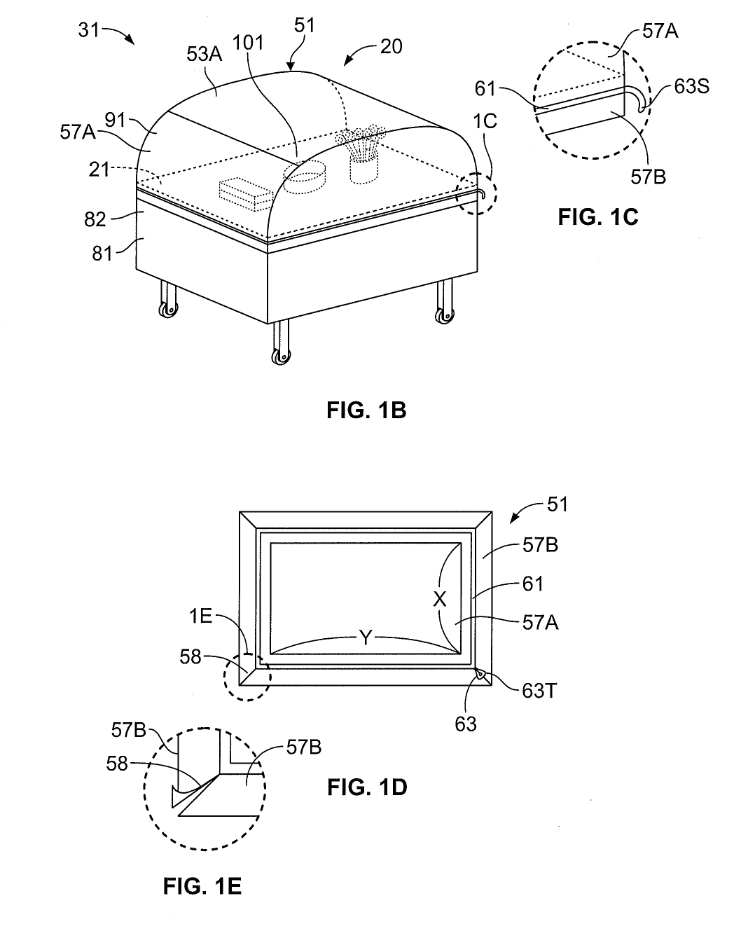

The embodiment of the system 31 illustrated in FIG. 1A includes a protective cover 51 and an attachment component 71. The illustrated embodiment of the protective cover 51 is formed as a continuous sheet 53 having an outer surface 53A and an inner surface 53B. The protective cover 51 is securable relative to the operational surface 21 through the use of the attachment component 71. The embodiment of the system 31 shown in FIG. 1A includes an attachment component 71 that is securable as an adhesive securing element 73. The adhesive securing element can be sized and shaped as needed to secure the protective cover to or adjacent to the operational surface 21. The adhesive securing element 73 of the embodiment illustrated in FIG. 1A includes an adhesive layer 74 positioned on the inner surface 53B and in the lower portion 57B of the protective cover 51. The adhesive layer 74 shown in FIG. 1A is sized and shaped to permit the protective cover 51 to be secured to each of the generally vertical surfaces 121V, 122V, 123V, 124V of the table 20. The adhesive securing element 73 includes a removable element 75 that protects and prevents the exposure of the adhesive of the adhesive layer 74 until a user wishes to secure the protective cover 51 relative to the operational surface 21. FIG. 2A shows a portion of the adhesive securing element 73 in which the removable element 75 is lifted to show the adhesive layer 74. As so secured, the protective cover 51 provides a protective interior 91 in which the items 101 positioned relative to the horizontal surface 20H of the table 20 are protected from additional exposure to “ambient conditions” defined for purposes of this application as conditions that exist at, above, and exterior to the outer surface 53A of the protective cover 51. Such conditions may be non-sterile conditions, or contaminated conditions, or contain one or more sources from which contaminants may be produced.

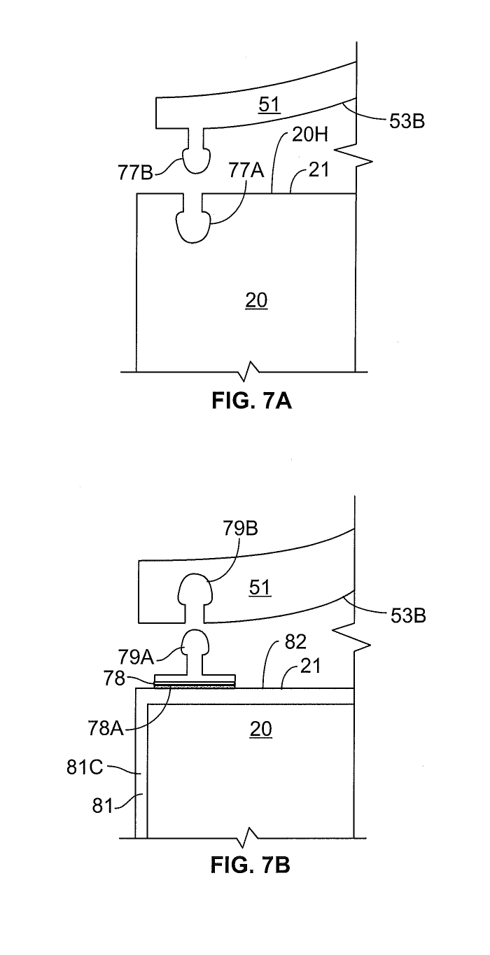

FIG. 7A shows an embodiment of the present invention by which at least partial securement of the protective cover 51 to the operational surface 21 may be accomplished through a mechanical connection. In the illustrated embodiment, the surface 20H of the table 20 includes a female element 77A in which an appropriately configured male element 77B formed on or as part of the inner surface 53B of the protective cover 51. With such an attachment component 71, the protective cover 51 can be opened and closed as needed.

To provide additional protection for that which is positioned on or relative to the operational surface 21, an additional cover 81 may be used. One embodiment of the additional cover 81 is illustrated in FIGS. 1B and is a cover element 81C of the type that has been conventionally used as an under cover for items 101 placed, for example, on the horizontal surface 20H of a table 20 in preparation for a surgical procedure. The cover element 81C is sized and shaped to cover at least the horizontal surface 20H. The embodiment of the cover element 81C shown in FIG. 1B is sized and shaped to cover not only the horizontal surface 20H but also, by draping vertically, the sides 121V, 122V, 123V, 124V of the table 20. Such coverage of at least a portion of the sides of the table by a sterile cover 81 allows the table to be touched or moved during the course of, for example, surgery with some confidence that such action did not contaminate the user. Certain embodiments of the protective cover 51 may be secured to the upper surface 82 of the cover element 81C by removing the removable element 75 and pressing the exposed adhesive layer 74 onto the surface 82. When so secured, the protective cover 51 provides a protective interior 91 in which the items 101—positioned relative to the horizontal surface 20H of the table 20 and on at least a portion of the upper surface 82 of the cover element 81C—are protected from additional exposure to ambient conditions.

FIG. 7B illustrates an embodiment of the system 31 in which the attachment component includes a tape component 78 having an adhesive element 78A by which the tape 78 may be secured directly to the operational surface 21 or to a cover 81 placed on the operational surface 21. The tape component 78 includes the portion of a mechanical connector—in the illustrated embodiment, a female tape element 79A—that corresponds to the portion that is formed in or on the protective cover 51—in the illustrated embodiment, a male cover element 79B. By pressing the male cover element 79B onto the female tape element 79A, the protective cover 51 may be secured to the cover 81 and thereby the operational surface 21. The cover 51 thereby may be opened and closed as needed.

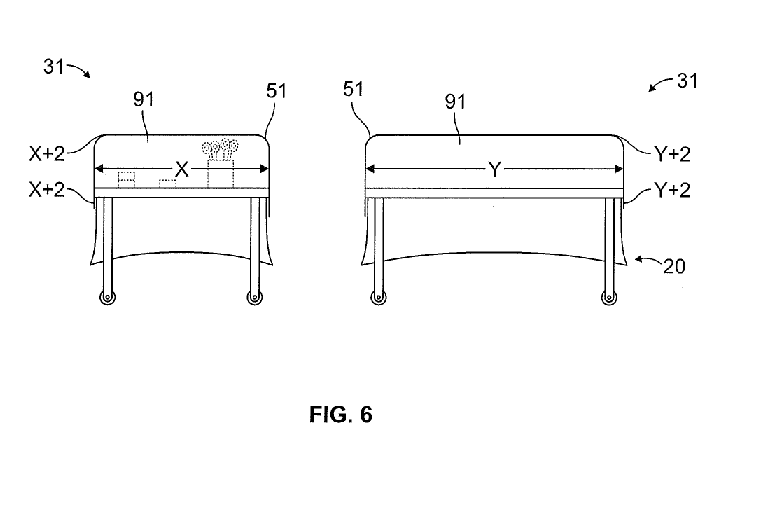

FIG. 6 shows embodiments of the protective cover 51 deployed relative to different sized tables 20 and over different sized operational surfaces 21. The embodiments shown in FIG. 6 include protective covers 51 whose overall size is larger than but in proportion to the operational surface 21 on and over which each is deployed. Through such configurations, an interior 91 is developed that is appropriate for the size and shape of the operational surfaces 21 and the items 101. By way of example only, a small size may be about 15″ larger than the table 10 on each side; a medium size may be about 25″ larger than table 10 on each side; and a large size may be about 35″ larger than table 10 on each side. For example, X (shorter side) by Y (longer side) size surgery instrument table: (a) small size-[X+31 inch: (15.5×2)]×[Y+31 inch: (15.5×2)]; (b) medium size-[X+51 inch: (25.5×2)]×[Y+51 inch: (25.5×2)]; and (c) large size-[X+71 inch: (35.5×2)]×[Y+71 inch: (35.5×2)]. In these embodiments, a 0.5″ extension beyond the side of the table is shown to allow for adequate coverage of all four sides of the table.

In certain applications in which the protective cover 51 is intended to be secured and opened once, the adhesive used to form the adhesive layer 74 may vary, and includes, but is not limited to, drying adhesives (solvent based and polymer dispersion adhesives), pressure sensitive adhesives, contact adhesives, and other synthetic adhesives such as epoxy, polyurethane, cyanoacrylate and acrylic polymers. In other applications in which the protective cover is intended to be secured, and possibly opened and closed many times, the adhesive used to form the adhesive layer 74 may be a “low-tack”, reusable, pressure-sensitive adhesive. Such an adhesive allows the protective cover to be stuck to an object (including a surface 20H of a table 20 or a bench), easily lifted off the object, and re-stuck as needed.

Certain embodiments of the present invention provide a protective cover 51 that will be opened only once and that readily inform the user whether the protective cover has been opened. For example, if the protective cover 51 is used to maintain sterile conditions of surgical instruments and materials 101 positioned on a surgical table, the protective cover 51 will be removed once. It is important in this application to know whether the protective cover 51 has been disturbed and the surgical instruments 101 exposed to possibly non-sterile ambient conditions. For such an application, certain embodiments of the adhesive layer may be configured to visually inform a user that the adhesive layer has been disturbed and that the protective interior 91 possibly exposed to ambient conditions. To signal such disturbance of the adhesive layer, the adhesive 74 may change in color (e.g., from clear to blue) at least at a portion of the protective cover at which the disturbance has occurred. It can be appreciated that other tamper indicators may be used, such as paint, tape, or labels. For example, a tamper proof tape that leaves a permanent residue on the surface from which it is being removed may also be used. Examples of the type of tape used in certain embodiments include the SafeLabel™-HR type tape manufactured by TamperSeals Group®. Examples of additional tamper indicators include those disclosed in U.S. Pat. Nos. 4,509,196; 4,557,505; 4,652,473; 4,980,222; 5,082,702; 5,518,763; 5,770,283; and 6,395,376 and U.S. Publication Nos. 2002/0142121; 2007/0071968; 2010/0285398; and 2012/0244623, all of which are incorporated herein by reference in their entirety. The tamper indicator may or may not be sterilized depending on the application.

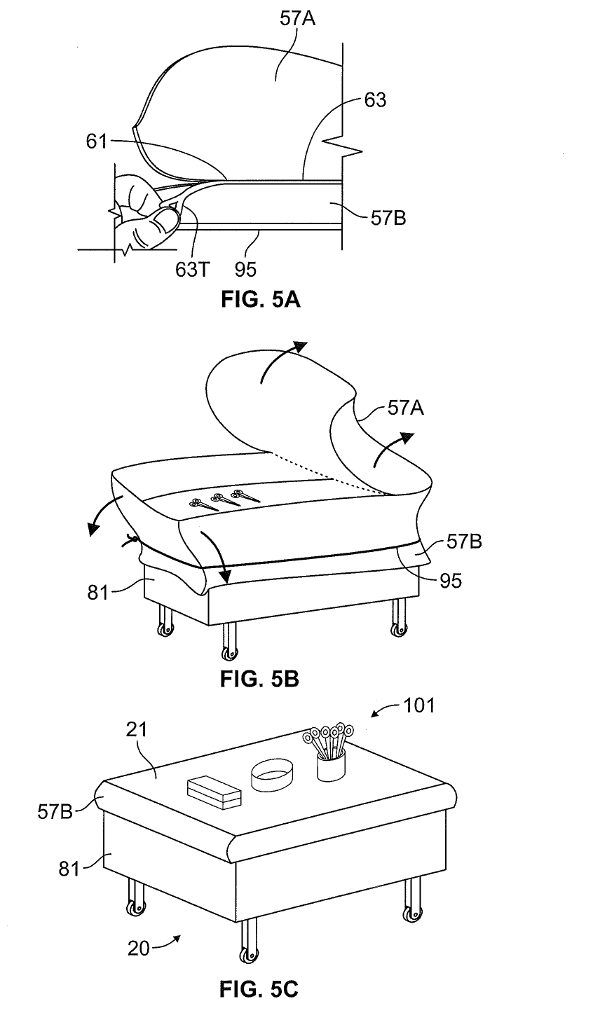

To allow a user to gain access to the protective interior 91, certain embodiments of the system 31 may include a tear portion 61. In certain embodiments of the present invention, the system 31 includes a tear portion 61 positioned such that at least a portion of the protective cover 51 may be separated from the portion by which attachment relative to the operational surface 21 may be accomplished. FIG. 1A through FIG. 1E and FIG. 5A illustrate certain embodiments of the system 31 that include a tear portion 61. In these illustrated embodiments, the tear portion 61 is disposed around the entire circumference of the protective cover 51. However, it is appreciated that embodiments of the present invention may include a tear portion 61 disposed along a select portion of the cover 51 (e.g., along two perpendicular sides, or along two adjacent sides) or that the system 31 include multiple tear portions 61 that may be used to gain access to certain portions of the interior 91.

In these embodiments, by operation of the tear portion 61, the protective cover 51 can be opened to form a top portion 57A and a lower portion 57B. A weakness in the material from which the protective cover 51 is formed may define the tear portion 61 and by operation of the tear element 63 the section at which the top portion 57A is openable relative to the lower portion 57B.

One embodiment of such a tear element 63 is a string 63S positioned within the material at least in part defining the tear portion 61. FIG. 1B illustrates such a tear element 63. When pulled by a user, the top portion 57A of the protective cover 51 opens relative to the lower portion 57B.

Another embodiment of the tear element 63 may include a tab 63T in the protective cover 51 that by pulling causes the protective cover 51 to tear. A weakness in the material used to form the protective cover 51 may make it easier to operate the tear element 63 and provide direction to the tearing.

The protective cover may be made from a material that provides the protective coverage sought for the operational surface 21 and the items 101 positioned relative to it. In order to maintain sterility of the interior 91, the entire protective cover 51 or the top portion 57A may be a clear cover made out of single or multiple layers of plastic or plastic-like material, such as polycarbonate, a composite, or any other material that is capable of maintaining sterility. Examples of an embodiment of the top portion include the Tiburon® Femoral Angiography Drape, which includes clear side panels. The protective cover 51 may be formed also by the extrusion of thermoplastic material as a continuous single or multi-ply web. The material from which the cover 51 is formed may be a multi-layer impermeable film, such as EVOH ply adhesively secured between polypropylene and low density polyethylene piles.

FIG. 1C provides a top view of one embodiment of the protective cover 51. In the embodiment shown here, the protective cover 51 has a rectangular shape (with the “Y” length in this embodiment greater than the “X” length). However, it can be appreciated that the cover 51 can have a square shape, circular shape, or any shape that is suitable by which protective coverage can be provided for an operational surface 21 and the items 101 positioned on it. As shown in FIG. 1D, FIG. 1E, and FIG. 2B, the bottom portion 57B may be configured to be complimentary so as to form one or more cover corners 58 that correspond to the one or more table corners 221C, 222C, 223C, 224C. Such complimentary correspondence aids in the maintenance of non-ambient conditions within the interior 91.

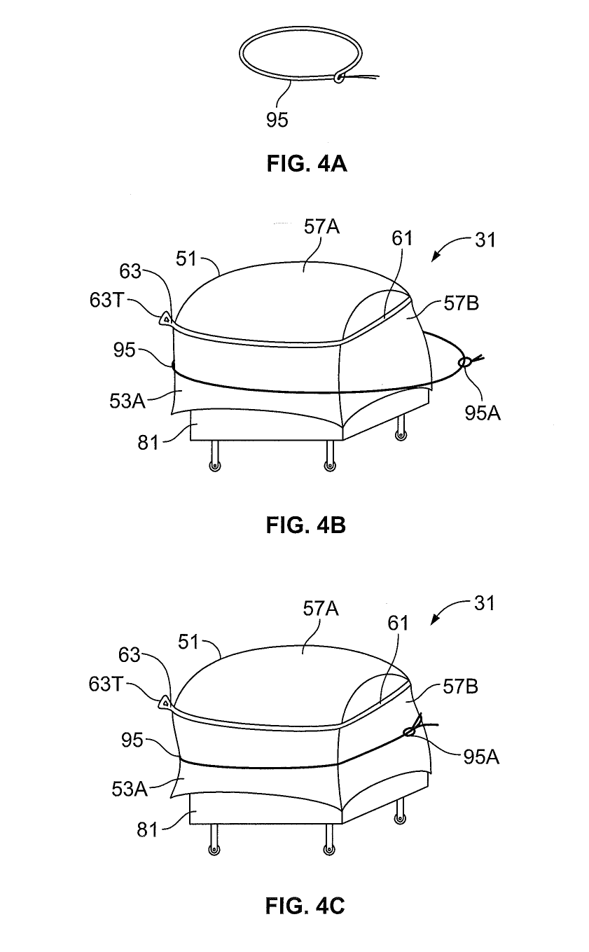

FIG. 4A, FIG. 4B, and FIG. 4C and FIG. 5A and FIG. 5B illustrate additional embodiments of the system 31 that include a restraint 95. The restraint 95 may be a security string/band that is sized and shaped and configured to be extendable around part or all the outer circumference of at least a portion of the outside surface 53A of the protective cover 51 and secure it relative to the operational surface 21. Such a restraint 95 may facilitate the development of a outer surface 53A of the seal by the protective cover in order to maintain, for example, the sterility of the surgical instruments 101 placed on the operational surface 21 or the freshness of food items or to prevent their contamination. The restraint may be used as the attachment component 71 for the protective cover 51 or in conjunction with another attachment component 71 such as a securing element 73. The restraint 95 may be made from any suitable material by which engagement with the protective cover 51 can be accomplished including fiber, plastic, silastic, rubber, cloth or the like. The restraint may include a tamper-proof lock 95A. FIG. 4A shows one such embodiment of such a restraint 95. FIG. 4B shows the embodiment of the restraint 95 shown in FIG. 4A in place around the lower portion 57B of the protective cover 51. FIG. 4C shows the embodiment of the restraint 95 shown in FIG. 4A and FIG. 4B tightened to assist in securing the protective cover 51 relative to the operational surface 21 with the tamper-proof lock 95A engaged.

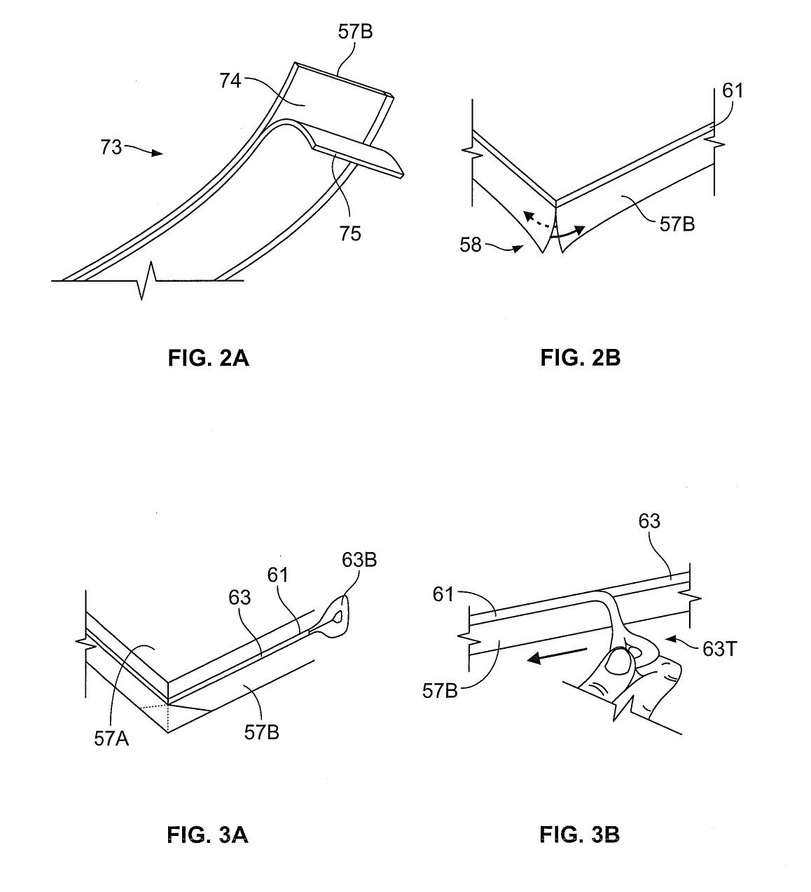

The following describes the operation of the embodiments of the system 31 that include a tear portion 61. A user by grabbing, for example, the string 63S shown in FIG. 1B or the tab 63T shown in FIG. 3B and FIG. 5A can disrupt the tear portion 61 and open the protective cover 51 to form a top portion 57A and a lower portion 57B. FIG. 5B illustrates an embodiment of the protective cover 51 in which the cover is made from a flexible material. By the operation of the tear portion 61, the lower portion 57B of the protective cover 51 can drop and/or be manipulated to drape over the sides 121V, 122V, 123V, 124V of the table 20 The restraint 95 maintains the lower portion 57B in position relative to the illustrated table 20. Such embodiments provide additional protection for the users during a procedure as explained above.

In certain embodiments, the protective cover 51 is provided within suitable packaging. The protective cover and other components may be provided as a kit, optionally containing certain items needed for the anticipated procedure. Depending on the application, the protective cover can be sterilized for medical applications—for example, chemically, by heat, or by radiation—or may be washable and possibly reusable for food or other applications. It will be apparent to one skilled in the art that the sterilization method and cover components must be compatible. In certain kit embodiments, the surgical cover can be sterilized together with surgical instruments.

1. A system for controlling exposure of one or more objects to ambient conditions, comprising,
a protective cover including a generally continuous sheet portion having an outer surface and an inner surface; and
an attachment component including an adhesive surface configured and of an adhesive composition such that said adhesive surface is securable to an operational surface of at least one of the one or more objects to define a protective interior in which non-ambient conditions can be maintained.

2. The system according to claim 1 in which said attachment component is positioned on said inner surface of said protective cover.

3. The system according to claim 1 in which said protective cover is made from a material and configured and said attachment composition is of a composition and configured to permit sterile conditions to be maintainable within said protective interior.

4. The system according to claim 1 further including a tear portion formed in said protective cover and configured to permit a user to open the protective cover and expose at least a portion of the operational surface

5. The system according to claim 1 in which said adhesive composition changes color at least at a portion of said protective cover that is removed from the operational surface.

6. The system according to claim 1 in which said adhesive composition is of a low-tack that allows said protective cover to be opened and closed relative to the operational surface more than once.

7. A system for preventing contamination of one or more objects, comprising,
a protective cover including a generally continuous sheet portion having an outer surface and an inner surface; and
an attachment component configured to permit the mechanical connection of said protective cover to an operational surface of at least one of the one or more objects to define a protective interior to prevent the contamination of the one or more objects.

8. The system according to claim 7, wherein said attachment component include a female element and a male element joinable to define said protective interior.

9. The system according to claim 8, wherein said male element or female element is formed in the operational surface.

10. The system according to claim 8, wherein said male element or female element is formed within a tape component, said tape component including an adhesive element by which said tape component may be securable to the operational surface.
